(19) United States
(12) Patent Application Publication
(10) Pub. No.: US 2020/0222353 A1
(43) Pub. Date: Jul. 16, 2020

(54) Ketone ester and ketone salt compositions and methods for using the same

(71) Applicant: Christopher David Thompson, Santa Monica, CA (US)

(72) Inventor: Christopher David Thompson, Santa Monica, CA (US)

(21) Appl. No.: 16/247,513

(22) Filed: Jan. 14, 2019

(51) Int. Cl. A61K 31/22; A61K 31/20

(52) U.S. Cl. CPC A61K 31/22; A61K 9/0053; A61K 31/20

(57) ABSTRACT

A composition comprising a combination of sodium beta-hydroxybutyrate, magnesium beta-hydroxybutyrate, potassium beta-hydroxybutyrate, calcium beta-hydroxybutyrate, and at least one ketone ester for oral administration to humans and other animals, as well as a method for administering said composition for enhanced physical or cognitive capabilities, physical endurance, or post-exercise recovery are described. The composition provides electrolytes to replace electrolytes lost during intense or extended physical activity to reduce the likelihood of cramps, muscle spasms, or fatigue from depletion of electrolytes. The composition concurrently provides ketone bodies, which raise the level of ketone bodies in the bloodstream and induce ketosis (>0.5 mM of ketone bodies in the bloodstream) and provide an alternative energy source to glycogen during physical activity. The composition generally does not cause side effects or discomfort in the user. The composition may optionally comprise at least one medium-chain triglyceride (MCT).

### CROSS-REFERENCE TO RELATED APPLICATIONS

Not applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

The field of the invention pertains to compositions comprising ketone salts and ketone esters, as well as methods for using the same.

The depletion of electrolytes, such as sodium, magnesium, potassium, and calcium, and of glycogen during physical exercise are among the major hindrances to the physical performance and physical endurance of humans. Electrolytes are typically lost through sweat and urine during exercise (Shirreffs et al., (2004) J. Sports Sci. 22(1):57-63). Athletes that do not replace electrolytes during physical exercise may experience an earlier onset of fatigue, muscle spasms, or muscle cramps, and therefore may be less able to perform the exercise. For example, loss of potassium during exercise may cause problems for the heart (Lindinger, (1995) J. Mol. Cell. Cardiol. 27(4):1011-1022) and have negative effects on muscle contraction (Lindinger and Sjogaard, (1991) Sports Med. 11(6):382-401). Low levels of calcium may result in reduced muscle contraction. Loss of magnesium can cause muscle cramping (Sinclair and Geiger, (1999) Sport Medicine Council of Manitoba, February: 1-4). Consequently, it is important to prevent low levels of electrolytes in the body during exercise and to replenish lost electrolytes after exercise.

Depletion of glycogen may also occur during physical exercise, even during mild physical exercise (Bonen et al., (1985) J. Appl. Physiol. 58(5):1622-1629). Depletion of glycogen is known to be correlated with an individual's resistance to fatigue during exercise, such that individuals reach exhaustion sooner when they have depleted reserves of glycogen (Green (1991) Can. J. Physiol. Pharmacol. 69(2):290-297).

Some common ways for replenishing glycogen and electrolytes to the body during exercise are the consumption of sport drinks like Gatorade®, energy gels like Honey Stinger, CLIF SHOT®, or GU™, and energy chews from GU™. These methods typically provide simple sugars and other simple carbohydrates to replace depleted glycogen, along with providing electrolytes.

Ketone bodies are a set of chemical compounds that may be metabolized as an alternative to glycogen. When ketone bodies are available in the bloodstream, the body may metabolize the ketone bodies before glycogen, such that glycogen stored in muscle tissue is preserved during exercise. Ketone bodies have also been shown to produce more work per amount of oxygen consumed (Sato et al. (1995) FASEB J. 9:651-658). Therefore, metabolism of ketone bodies may permit longer and more intense physical activity.

The liver naturally produces ketone bodies by breaking down adipose tissue when the body is in a state of “ketosis,” which is characterized by heightened levels of ketone bodies in the bloodstream. Achieving ketosis naturally, however, typically requires a regimen of intermittent fasting, physical exercise, and dieting according to a “ketogenic” diet. A ketogenic diet severely limits carbohydrate intake and requires that the majority of caloric intake come from fats. Achieving ketosis naturally may require weeks of strictly maintaining such a regimen; the consumption of a few tens of grams of additional carbohydrates can erase the progress made toward ketosis. Achieving ketosis naturally is therefore impractical for many individuals. Additionally, those who achieve ketosis naturally sometimes experience gastrointestinal distress as a result of following a ketogenic diet (Cai et al. (2017) World J. Pediatr. 13(6):528-536).

An alternative method to achieving ketosis is taking “exogenous ketones.” Exogenous ketones are compounds comprising ketone bodies or precursors thereof that may be consumed to raise the levels of ketone bodies in the bloodstream. While exogenous ketones are frequently used as an artificial means of reaching ketosis, individuals who have reached ketosis naturally may also take exogenous ketones to further increase the levels of ketone bodies in their bloodstreams.

A variety of exogenous ketones have been synthesized and studied, but each has disadvantages that the user must suffer to take an effective dose. One type of exogenous ketone is ketone salts. Ketone salts are powders comprising the conjugate base of a ketone (typically D-beta-hydroxybutyrate) chemically bound to a mineral salt. Consumption of an effective dose of a ketone salt necessarily involves the consumption of a large quantity of the mineral salt, which can lead to gastrointestinal discomfort and future health complications, such as hypertension in the case of excess sodium. Another type of exogenous ketone is ketone ester. A ketone ester is a compound containing ketone bodies that are linked chemically by ester bonds. While some ketone esters can be taken in effective doses without substantial side effects, such ketone esters are produced through costly chemical synthesis, and thus are much more expensive than other exogenous ketones. High levels of ketone bodies can also be achieved by taking ketones directly. Ketones are the compounds into which ketone bodies are broken down, examples of which include but are not limited to beta-hydroxybutyric acid (BHB) and acetoacetate (AcAc). Consuming ketones directly, however, may cause acidosis when the ketones are absorbed, do not elevate ketone body levels in the bloodstream for as long of a time as other exogenous ketones, and tend to be among the more expensive exogenous ketones. A precursor to ketone bodies, medium-chain triglycerides (MCTs), which are triglycerides having fatty acids containing aliphatic chains with 6-12 carbon atoms, may also be consumed to reach ketosis, though it is not a proper ketone body. MCTs, however, frequently cause gastrointestinal distress upon consumption of an effective dose. Consequently, the aforementioned methods for achieving ketosis artificially are not suitable for many users.

### BRIEF SUMMARY OF THE INVENTION

The present invention is a composition comprising at least one of the following three ketone esters, [(3R)-3-hydroxybutyl] (3S)-3-hydroxybutanoate, [(3S)-3-hydroxybutyl](3R)-3-hydroxybutanoate, and [(3S)-3-hydroxybutyl] (3S)-3-hydroxybutanoate alone or with any of the mineral based beta-hydroxybutyrates [sodium beta-hydroxybutyrate, magnesium beta-hydroxybutyrate, potassium beta-hydroxybutyrate, calcium beta-hydroxybutyrate], a combination of sodium beta-hydroxybutyrate, magnesium beta-hydroxybutyrate, potassium beta-hydroxybutyrate, calcium beta-hydroxybutyrate, and at least one of the following three ketone esters, [(3R)-3-hydroxybutyl] (3S)-3-hydroxybutanoate, [(3S)-3-hydroxybutyl] (3R)-3-hydroxybutanoate, and [(3S)-3-hydroxybutyl] (3S)-3-hydroxybutanoate. The present invention is designed to provide both the electrolytes required for healthy functioning of the user and the ketone bodies required for the user to reach ketosis, while having a high tolerability for the user. Consumption of the disclosed composition may help to preserve glycogen levels in muscle tissue and replenish electrolytes lost during exercise, particular after intense or extended physical activity. Consumption of the disclosed composition may also help to enhance the physical performance, physical endurance, and post-exercise recovery of the user during the time period over which the disclosed composition is effective.

In one embodiment of the present invention, the disclosed composition additionally comprises at least one type of MCT. The MCTs in this embodiment may increase ketone levels in the bloodstream of the user and provide additional calories from fat, but they are not required for the proper functioning of the present invention.

In addition to providing electrolytes and ketones in the bloodstream, the disclosed composition generally possesses several features that are generally preferred among users. In a preferred embodiment, the composition of is balanced to reduce the problems associated with larger amounts of its main components. These problems comprise, but are not limited to, an unhealthy excess of mineral salts from the consumption of an effective dose of ketone salts, the prohibitive cost of an effective dose of ketone esters, and gastrointestinal distress caused by an effective dose of MCTs. As a result, this preferred embodiment of the present invention is less expensive than ketone esters and has a higher tolerability for the user than pure ketone salts or MCTs.

In a further embodiment, the disclosed composition is administered orally in admixture with a carrier or excipient comprising other components that may be inactive or active. These components are intended to be, but are not limited to being, non-toxic and not greatly suppressing the levels of ketone bodies in the bloodstream of the user.

In an additional embodiment, the present invention is also a dietary supplement comprising the disclosed composition for oral administration.

In a still further embodiment, the present invention is administered before physical exercise to provide electrolytes that will be lost during the exercise, such that healthful levels of electrolyte are maintained, as well as to raise levels of ketone bodies in the bloodstream.

In another embodiment, the present invention is administered during physical exercise to maintain healthful levels of electrolytes and to raise levels of ketone bodies in the bloodstream.

In yet another embodiment, the present invention is administered after physical exercise to replenish electrolytes that were lost during the exercise to healthful levels and to raise levels of ketone bodies in the bloodstream.

### BRIEF DESCRIPTION OF THE SEVERAL VIEWS OF THE DRAWING

FIG. 1 shows the chemical structure of [(3R)-3-hydroxybutyl] (3S)-3-hydroxybutanoate.

FIG. 2 shows the chemical structure of [(3S)-3-hydroxybutyl] (3R)-3-hydroxybutanoate.

FIG. 3 shows the chemical structure of [(3S)-3-hydroxybutyl] (3S)-3-hydroxybutanoate.

### DEFINITIONS

The abbreviation “BHB” refers to either of the enantiomers of beta-hydroxybutyric acid (IUPAC name: 3-hydroxybutanoic acid), which are (3R)-3-hydroxybutanoic acid and (3S)-3-hydroxybutanoic acid.

The abbreviation “D-BHB” refers specifically to the “R” enantiomer of BHB, (3R)-3-hydroxybutanoic acid.

The abbreviation “L-BHB” refers specifically to the “S” enantiomer of BHB, (3 S)-3-hydroxybutanoic acid.

The term “ketone ester” refers to any chemical compound containing at least one ester bond which, when the ester bond(s) is broken in a hydration reaction, the resulting compounds include at least one ketone body. The three ketone esters considered primarily in this patent are [(3R)-3-hydroxybutyl] (3S)-3-hydroxybutanoate, [(3S)-3-hydroxybutyl] (3R)-3-hydroxybutanoate, and [(3S)-3-hydroxybutyl] (3S)-3-hydroxybutanoate.

The term “medium-chain triglyceride” or “MCT” refers to any triglyceride comprising fatty acids having an aliphatic tail of 6-12 carbon atoms.

The term “ketone salt” refers to any compound comprising, beta-hydroxybutyrate (IUPAC name: 3-hydroxybutanoate) and a mineral salt or amino acid, which may comprise but is not limited to, sodium, magnesium, calcium, potassium, or other mineral salts or amino acids.

The term “ketone body” refers to a chemical compound that may be produced from chemical reactions involving adipose tissue and occurring in the liver, and which may be broken down into at least one molecule of a ketone.

The term “ketone” refers to any of the three dietary ketones, beta-hydroxybutyrate, acetoacetate, and acetone.

The term “physical performance” refers to the degree or extent to which physical activities, often competitive, may be performed, some non-limiting examples being the time required to run, cycle, swim, or a given distance, the accuracy of shots in basketball, soccer, or hockey, or the completion of routes in rock-climbing, bouldering, or the degree of performance of other physical activities.

The term “physical endurance” refers to the duration of time or number of repetitions for which physical activities, often competitive, may be performed. Some non-limiting examples are the number of push-ups, pull-ups, sit-ups, or chin-ups that can be performed before exhaustion, the distance that may be hiked, run, swam, rowed, skated, or cycled before exhaustion, the length of play time in hockey, soccer, American football, basketball, or other such sport before requiring a substitute player due to exhaustion, or extent of other physical feats that can be reached before exhaustion.

The term “post-exercise recovery” refers to the process by which muscles are repaired and nutrients replenished after physical exercise. Typical, but not necessary, results of post-exercise recovery include, but are not limited to, reduced soreness and preparation of muscles for future exercise.

The term “ketosis” refers to the state of a body in which the levels of ketone bodies in the bloodstream exceed 0.5 mM.

The term “effective dose” refers to the minimum threshold for the dose of a compound, such that doses exceeding this threshold in quantity are sufficient to raise the levels of ketone bodies in the bloodstream of the user above 0.5 mM and inducing ketosis in the user. The effective dose of a substance may be specific to the substance as well as to the user, and therefore may vary for different substances as well as for different users. The effective dose for a user may be determined on the basis of experimentation or through predictions based on empirical evidence. For a substance to which a user may develop tolerance, the effective dose may increase following regular consumption of the substance.

The term “user” refers to a human or other animal, at least one of whose physical performance, physical endurance, or post-exercise recovery may be enhanced upon receiving the disclosed composition through oral administration.

The term “tolerability” refers to the lack of aversion of the body to the consumption of a compound. Compounds with low tolerability may cause discomfort or more severe problems in the user, such as, but not limited to, gastrointestinal distress, electrolyte imbalance, vomiting, nausea, or other unpleasant side effects. Compounds with high tolerability cause the user minor or no noticeable side effects upon consumption.

The term “healthful levels” refers to levels of electrolytes that promote the health of the user. Healthful levels of electrolytes may vary among users, and may vary as a result of age, sex, physical activity, or personal characteristics. Healthful levels may be determined on the basis of experimentation or through predictions based on empirical evidence.

### DETAILED DESCRIPTION OF THE INVENTION

In accordance with the present invention, a composition comprising sodium beta-hydroxybutyrate, magnesium beta-hydroxybutyrate, potassium beta-hydroxybutyrate, calcium beta-hydroxybutyrate, and at least one of the following three ketone esters, [(3R)-3-hydroxybutyl] (3S)-3-hydroxybutanoate, [(3S)-3-hydroxybutyl] (3R)-3-hydroxybutanoate, and [(3S)-3-hydroxybutyl] (3S)-3-hydroxybutanoate, is orally administered to a user to increase the levels of the electrolytes sodium, potassium, calcium, and magnesium in the user to healthful levels, as well as to increase the levels of ketone bodies in the bloodstream of the user above 0.5 mM. The combination of ketone salts and at least one ketone ester, as well as optionally at least one MCT, results in enhancement in the physical performance, physical endurance, and post-exercise recovery of the user upon consumption while having a high tolerability for the user as compared to the effective dose of any individual component. These competing goals are not simultaneously achievable with any single component. By concurrently providing these nutrients in the ratios hereafter described, the disclosed composition may also enhance mental focus by reducing bodily distractions caused by inadequate nutrition, providing nutrients required for proper mental functioning, and simplifying the potentially complex and stressful process of acquiring such nutrients in appropriate quantities. Resulting enhancements in mental focus from consumption of the disclosed composition may further enhance physical performance due to the requirement of mental focus to perform some physical activities to the best of the user's abilities.

Each ketone salt in the disclosed composition co-delivers its mineral salt along with a ketone body, that is, sodium beta-hydroxybutyrate co-delivers sodium, magnesium beta-hydroxybutyrate co-delivers magnesium, potassium beta-hydroxybutyrate co-delivers potassium, and calcium beta-hydroxybutyrate co-delivers calcium. The amount of each ketone salt is determined to help the user reach healthful levels of sodium, magnesium, potassium, and calcium.

An effective dose of ketone salts has been shown to raise the levels of BHB in the bloodstream above 0.5 mM after consumption in the form of a drink in human clinical trials (Rodger et al., (2017) J. Sci. Cycling 6(1):26-31; O'Malley et al., (2017) Appl. Physiol. Nutr. Metab. 42(10):1031-1035). Since the disclosed composition comprises additional sources of ketone bodies besides an individual ketone salt, less of each ketone salt is required in an effective dose of the present invention than is contained in an effective dose of an individual ketone salt. The disclosed composition may therefore have a higher tolerability than an effective dose of an individual ketone salt. Additionally, in an embodiment of the present invention that includes MCTs, the amount of MCTs may also be increased to raise the level of ketone bodies in the bloodstream yet higher, while not exceeding a dose with a low tolerability, which may cause gastrointestinal distress in the user upon consumption.

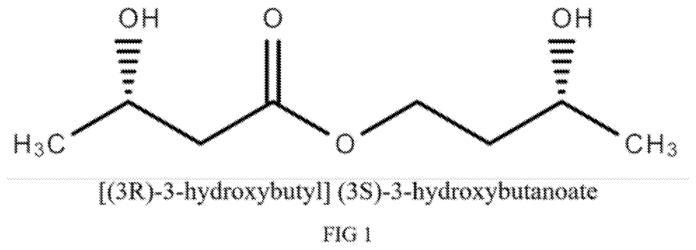

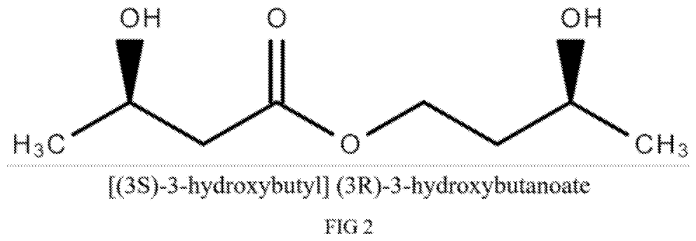

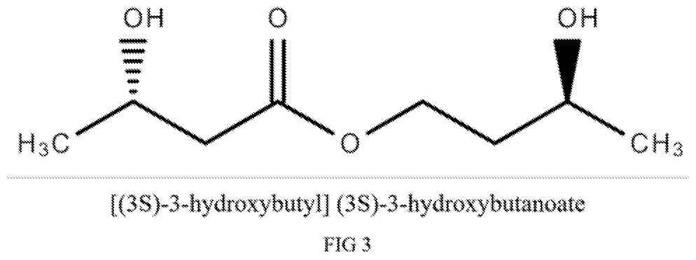

The ketone esters considered as part of the present invention, [(3R)-3-hydroxybutyl] (3S)-3-hydroxybutanoate, [(3S)-3-hydroxybutyl] (3R)-3-hydroxybutanoate, and [(3S)-3-hydroxybutyl] (3S)-3-hydroxybutanoate, are “ketone monoesters,” as they have a single ester bond between the two ketone bodies. [(3R)-3-hydroxybutyl] (3S)-3-hydroxybutanoate is shown in FIG. 1, [(3S)-3-hydroxybutyl] (3R)-3-hydroxybutanoate is shown in FIG. 2, and [(3S)-3-hydroxybutyl] (3S)-3-hydroxybutanoate is shown in FIG. 3. When decomposed through a hydration reaction, [(3R)-3-hydroxybutyl] (3S)-3-hydroxybutanoate is converted into one molecule of L-BHB and one molecule of butane-1,(3S)-diol; [(3S)-3-hydroxybutyl] (3R)-3-hydroxybutanoate is converted into one molecule of D-BHB and one molecule of butane-1,(3R)-diol; and [(3S)-3-hydroxybutyl] (3S)-3-hydroxybutanoate is converted into one molecule of L-BHB and one molecule of butane-1,(3S)-diol. L-BHB has been shown not to be broken down in the body and used for energy as quickly as D-BHB, for reasons still unknown (Stubbs et al., (2017) Front. Physiol. 8:848). Preferred embodiments of the present invention therefore comprise a greater proportion of [(3S)-3-hydroxybutyl] (3R)-3-hydroxybutanoate. Nevertheless, L-BHB and its precursors provide an alternative energy source to glycogen during exercise, and therefore may assist in reducing the depletion of glycogen, which may enhance the physical performance, physical endurance, and post-exercise recovery of users, though possibly to a lesser extent than D-BHB and its precursors.

In a preferred embodiment, the composition is particularly intended to enhance physical performance, physical endurance, and post-exercise recovery, although the composition may also enhance mental capabilities since D-BHB, one of the byproducts of the metabolism of ketone salts and of some of the ketone esters considered, has been shown to cross the blood-brain barrier (BBB) and is a source of energy for neurons (Murray et al., (2016) FASEB J. 30(12):4021-4032).

The present invention may enhance physical performance, physical endurance, and post-exercise recovery for users following a ketogenic diet and intermittently fasting, as well as for users who are only maintaining a ketogenic diet, users who are only intermittently fasting, and users who are doing neither. If the user has elevated the levels of ketone bodies in his or her bloodstream through natural means, such as, but not limited to, following a ketogenic diet and intermittently fasting, the user may still benefit from the replenishment of electrolytes and additional supply of ketone bodies in the bloodstream that result from the consumption of the present invention. After consumption of the present invention, such a user may have higher levels of ketone bodies in his or her bloodstream than a user that has not elevated the levels of ketone bodies in his or her bloodstream through natural means. For both types of users, consumption of the present invention generally induces ketosis.

The disclosed composition generally has a high tolerability and does not cause the user to experience adverse side effects. Some studies have shown that consumption of doses above 0.38 g/kg bodyweight of a ketone salt may cause gastrointestinal distress during exercise (Evans et al., (2018) Eur. J. Sport Sci. 18(3):376-386; Fischer et al., (2018) J. Nutr. Metab. 2018:9812806), so preferred embodiments of the disclosed composition comprise less than 0.38 g/kg bodyweight of any individual ketone salt. Other studies have shown that daily consumption of 0.25-0.35 g/kg bodyweight of MCTs may result in gastrointestinal distress (Henderson et al., (2009) Nutr. Metab. (Lond) 6:31). Preferred embodiments of the disclosed composition therefore comprise less than 0.25 g/kg of MCTs. Studies on ketone esters have shown examples in which the ketone ester does not cause noticeable side effects when consumed in doses of 0.5 g/kg bodyweight (Cox et al., (2016) Cell Metab. 24(2):256-268). Preferred embodiments of the disclosed composition may therefore comprise at least up to 0.5 g/kg bodyweight of ketone esters.

The disclosed composition may be in admixture with a carrier or excipient. The carrier or excipient may comprise non-toxic compounds that may be active or inactive and that do not greatly suppress the levels of ketone bodies resulting from consumption of the disclosed composition. The carrier or excipient may also comprise compounds that stabilize the disclosed composition. Furthermore, during the period of time after production of the disclosed composition and before its consumption, the potential of the disclosed composition to enhance the physical performance, physical endurance, and post-exercise recovery of a user upon consumption may decline with time. As such, the carrier or excipient may slow this decline.

The carrier or excipient may also ease the consumption of the disclosed composition. As non-limiting examples, the carrier or excipient may ease consumption of the disclosed composition by bulking up the composition to increase its volume, by preventing clumping of powder particles if it is in the form of a powder, by reducing its viscosity for easier ingestion if it is in liquid form, or by increasing the solubility in a liquid for consumption in liquid form, as consumption in a liquid form may be easier for some users than consumption in a solid form. The carrier or excipient may also be selected to reduce gastrointestinal distress, which may, as a non-limiting example, be achieved by containing fewer compounds derived from milk or milk-derived substances, fewer dietary fibers, fewer fats, and more liquids and more simple sugars.

Furthermore, the carrier or excipient may enhance the visual appeal of the composition by adding color, such as with food coloring. The carrier or excipient may also enhance the taste of the composition, such as by adding natural or artificial flavorings.

The carrier or excipient may comprise, as non-limiting examples, sugar, starch, cellulose, powdered tragacanth, malt, gelatin, talc, cocoa butter, suppository wax, oil, mineral oil, food coloring, natural flavorings, artificial flavorings, citric acid, glycol, polyol, ester, agar, buffering agent, alginic acid, isotonic saline, Ringer's solution, ethyl alcohol, polyester, polycarbonate polyethylene glycol, polyvinylpyrrolidone, water, polyanhydride, antiadherents such as magnesium stearate, or other compound that is non-toxic and that does not greatly suppress the levels of ketone bodies in the bloodstream upon consumption of the disclosed composition.

The disclosed composition, along with any carrier or excipient that may be added to it, may be orally administered as a liquid, tablet, powder, troche, capsule, elixir, suspension, syrup, wafer, chewing gum, or food. Because the compound comprises ketone salts, which are more soluble in water than ketone esters, the compound is more easily dissolved in water than ketone esters or MCTs, and thus is more amenable to administration in as liquid in which the present invention is at least partially dissolved.

Preferred amounts of sodium beta-hydroxybutyrate within the disclosed composition are about 0.001 g/kg bodyweight to about 0.06 g/kg bodyweight, about 0.005 g/kg bodyweight to about 0.03 g/kg bodyweight, about 0.01 g/kg bodyweight to about 0.02 g/kg bodyweight, or any intervening range therein. In particular preferred embodiments, the disclosed composition comprises about 0.01 g/kg bodyweight, about 0.015 g/kg bodyweight, about 0.02 g/kg bodyweight, or any intervening amount of sodium beta-hydroxybutyrate therein.

Preferred amounts of magnesium beta-hydroxybutyrate within the disclosed composition are about 0.001 g/kg bodyweight to about 0.1 g/kg bodyweight, about 0.01 g/kg bodyweight to about 0.06 g/kg bodyweight, about 0.02 g/kg bodyweight to about 0.04 g/kg bodyweight, or any intervening range therein. In particular preferred embodiments, the disclosed composition comprises about 0.02 g/kg bodyweight, about 0.03 g/kg bodyweight, about 0.04 g/kg bodyweight, or any intervening amount of magnesium beta-hydroxybutyrate therein.

Preferred amounts of potassium beta-hydroxybutyrate within the disclosed composition are about 0.001 g/kg bodyweight to about 0.4 g/kg bodyweight, about 0.005 g/kg bodyweight to about 0.2 g/kg bodyweight, about 0.01 g/kg bodyweight to about 0.1 g/kg bodyweight, about 0.03 g/kg bodyweight to about 0.06 g/kg bodyweight, or any intervening range therein. In particular preferred embodiments, the disclosed composition comprises about 0.04 g/kg bodyweight, about 0.05 g/kg bodyweight, about 0.06 g/kg bodyweight, or any intervening amount of potassium beta-hydroxybutyrate therein.

Preferred amounts of calcium beta-hydroxybutyrate within the disclosed composition are about 0.001 g/kg bodyweight to about 0.1 g/kg bodyweight, about 0.01 g/kg bodyweight to about 0.06 g/kg bodyweight, about 0.02 g/kg bodyweight to about 0.04 g/kg bodyweight, or any intervening range therein. In particular preferred embodiments, the disclosed composition comprises about 0.02 g/kg bodyweight, about 0.03 g/kg bodyweight, about 0.04 g/kg bodyweight, or any intervening amount of calcium beta-hydroxybutyrate therein.

A particularly preferred recipe of ketone salts comprises a ratio of about 1:2:2:4 sodium beta-hydroxybutyrate:magnesium beta-hydroxybutyrate:potassium beta-hydroxybutyrate:calcium beta-hydroxybutyrate, with a total mass of ketone salt below 0.38 g/kg bodyweight, although the desired effects of the disclosed composition may be realized with embodiments comprising different ratios of the ketone salts in a different total amount.

Preferred percentages of ketone ester within the disclosed composition are about 0.1% to about 50%, about 0.5% to about 40%, about 2% to about 30%, about 5% to about 25%, about 10% to about 20%, or any intervening range therein. In particular preferred embodiments, the disclosed composition comprises about 10%, about 15%, about 20%, or any intervening percentage of ketone ester therein. The ketone ester may be a racemic mixture of the aforementioned enantiomers, may be enantiomerically enriched to comprise over 50% of one enantiomer, or may be purely (>90%) one enantiomer.

In one embodiment of the present invention, the composition further comprises at least one MCT. In this embodiment, the MCT(s) may be broken down into ketone bodies that may further raise the levels of ketone bodies in the bloodstream upon consumption of the present invention. The fat typically present in MCTs may also provide additional calories to the user. Not wishing to be bound by any particular theory or model, an advantage of this embodiment is that by further incorporating MCT into the present invention, the satiation experienced by the user upon consumption of the present invention may be extended due to the greater satiety experienced from consumption of calories derived from fat sources. Nevertheless, this advantage is not required for this embodiment.

The MCT(s) may comprise, for example, fatty acids with carbon chains of 6-12 carbon atoms, such as, but not limited to, caproic acid, caprylic acid, capric acid, and lauric acid. The MCT(s) may be derived from sources, such as, but not limited to, vegetable oil, glycerin, palm kernel oil, dairy products and coconut oil.

Preferred amounts of the MCT(s) within the disclosed composition are about 0.001 g/kg bodyweight to about 0.3 g/kg bodyweight, 0.005 g/kg bodyweight to about 0.2 g/kg bodyweight, 0.01 g/kg bodyweight to about 0.1 g/kg bodyweight, or any intervening range therein. In particular preferred embodiments, the disclosed composition comprises about 0.01 g/kg bodyweight, about 0.05 g/kg bodyweight, about 0.1 g/kg bodyweight, or any intervening amount of the MCT(s) therein.

The duration of ketosis in the user after consumption of an effective dose of preferred embodiments of the present invention are about 1 hour to about 12 hours, about 2 hours to about 8 hours, about 4 hours to about 6 hours or any intervening range therein. In particular preferred embodiments, the duration of ketosis after consumption of the disclosed composition in the user are about 4 hours, about 5 hours, about 6 hours, or any intervening duration of ketosis therein.

Preferred embodiments of the present invention induce ketosis in the user within about thirty minutes after consumption of an effective dose, although other embodiments may induce ketosis in the user within about one-hundred minutes, within about sixty minutes, or within about forty-five minutes after consumption of an effective dose. Particularly preferred embodiments of the present invention induce ketosis in the user within about fifteen minutes, within about ten minutes, or within about five minutes after consumption of an effective dose.

The disclosed composition has many advantages, some of which, but not all of which, are described here. Not all advantages are required by all embodiments of the composition.

This disclosure has described embodiments as examples, but has not described all possible embodiments of the composition or associated methods. Where a particular feature is disclosed in the context of a particular embodiment, that feature can also be used, to the extent possible, in combination with and/or in the context of other-embodiments. Insubstantial modifications of the invention, not presently foreseen, may nonetheless represent equivalents thereto.

What is claimed is:

1. A composition comprising at least one of the following three ketone esters, [(3R)-3-hydroxybutyl] (3S)-3-hydroxybutanoate, [(3S)-3-hydroxybutyl] (3R)-3-hydroxybutanoate, and [(3S)-3-hydroxybutyl] (3S)-3-hydroxybutanoate alone or with any of the mineral based beta-hydroxybutyrates [sodium beta-hydroxybutyrate, magnesium beta-hydroxybutyrate, potassium beta-hydroxybutyrate, calcium beta-hydroxybutyrate], and wherein said composition is in admixture with a carrier or excipient comprising a sugar, starch, cellulose, powdered tragacanth, malt, gelatin, talc, cocoa butter, suppository wax, oil, mineral oil, food coloring, natural flavorings, artificial flavorings, citric acid, glycol, polyol, ester, agar, buffering agent, alginic acid, isotonic saline, Ringer's solution, ethyl alcohol, polyester, polycarbonate polyethylene glycol, polyvinylpyrrolidone, water, polyanhydride, or magnesium stearate, and wherein said composition is formulated for oral administration.

2. The composition of claim 1 further comprising at least one type of medium-chain triglyceride (MCT), such as caproic acid, caprylic acid, capric acid, lauric acid, or other triglycerides comprising fatty acids having an aliphatic tail of 6-12 carbon atoms.

3. The composition of claim 1, wherein the formulation comprises a liquid, tablet, powder, troche, capsule, elixir, suspension, syrup, wafer, chewing gum, or food.

4. A method for administering a composition comprising sodium beta-hydroxybutyrate, magnesium beta-hydroxybutyrate, potassium beta-hydroxybutyrate, calcium beta-hydroxybutyrate, and at least one of the following three ketone esters, [(3R)-3-hydroxybutyl](3S)-3-hydroxybutanoate, [(3S)-3-hydroxybutyl] (3R)-3-hydroxybutanoate, and [(3S)-3-hydroxybutyl] (3S)-3-hydroxybutanoate, wherein said composition is in admixture with a carrier or excipient comprising a sugar, starch, cellulose, powdered tragacanth, malt, gelatin, talc, cocoa butter, suppository wax, oil, mineral oil, food coloring, natural flavorings, artificial flavorings, citric acid, glycol, polyol, ester, agar, buffering agent, alginic acid, isotonic saline, Ringer's solution, ethyl alcohol, polyester, polycarbonate polyethylene glycol, polyvinylpyrrolidone, water, polyanhydride, or magnesium stearate, wherein said composition is formulated for oral administration, and wherein the method comprises a regimen for the consumption of the composition in conjunction with physical activity for the purpose of enhanced physical performance, physical endurance, or post-exercise recovery.

5. The method of claim 4, in which the composition further comprises at least one type of medium-chain triglyceride (MCT), such as caproic acid, caprylic acid, capric acid, lauric acid, or other triglycerides comprising fatty acids having an aliphatic tail of 6-12 carbon atoms.

6. The method of claim 4, wherein the formulation of the composition comprises a liquid, tablet, powder, troche, capsule, elixir, suspension, syrup, wafer, chewing gum, or food.
